(19) United States
(12) Patent Application Publication
(10) Pub. No.: US 2019/0062835 A1
(43) Pub. Date: Feb. 28, 2019

(54) USE OF ALLELIC VARIANTS (SNPS) IN THE REGION 6P21.33 FOR THE DIAGNOSIS, PROGNOSIS AND TREATMENT OF MÉNIÈRE'S DISEASE

(71) Applicants: SERVICIO ANDALUZ DE SALUD, Sevilla (ES); FUNDACIÓN PÚBLICA ANDALUZA PROGRESO Y SALUD, Sevilla (ES)

(72) Inventors: José Antonio LÓPEZ ESCÁMEZ, Sevilla (ES); Sonia CABRERA MARTÍNEZ, Sevilla (ES); Marta Eugenia ALARCÓN RIQUELME, Sevilla (ES); María Teresa REQUENA NAVARRO, Sevilla (ES)

(21) Appl. No.: 15/766,800

(22) PCT Filed: Oct. 7, 2016
(86) PCT No.: PCT/ES2016/070716

§ 371 Date: Apr. 6, 2018

(30) Foreign Application Priority Data
Oct. 9, 2015 (ES) P201531458

(51) Int. Cl. C12Q 1/6883; C12N 15/10

(52) U.S. Cl. CPC C12Q 1/6883; C12Q 2600/118; C12N 15/1089

(57) ABSTRACT

The present invention relates to the use of a group of single-nucleotide polymorphisms or variants (SNPs) in chromosome 6 to obtain data that is useful in the prognosis of a disease involving sensorineural hearing loss, to a kit or devices and to the uses thereof.

The present invention is comprised in the field of biomedicine and biotechnology, and relates to a method of obtaining data that is useful for the prognosis and classification of patients suffering from sensorineural hearing loss, and particularly of those presenting Ménière's disease with bilateral involvement.

### PRIOR ART

Ménière's disease (MD) is a chronic disorder of the inner ear characterized by recurrent episodes of vertigo, progressive instability, pressure in the ears, tinnitus and hearing loss. MD is not only a disease but also a common clinical syndrome with several causes, such as heredity, immune response or allergic response, inter alia. MD can affect both ears in 10-40% of patients, which defines a significant impact on hearing, chronic disequilibrium and health-related quality of life. For this reason, bilateral MD is considered the most debilitating form of MD, and its therapeutic approach is different, including the use of immunosuppressants or a cochlear implant, thereby avoiding vestibular neurectomy. Given that an increase in the number of autoantibodies and a clinical improvement with steroid therapy are observed, an autoimmune etiology has been proposed for this disease. Some genetic variants in immune system genes such as MICA, TLR10 and NFKB1 seem to modify the clinical course of the disease, possibly as a result of an altered innate immune response.

Human Major Histocompatibility Complex class I and class II molecules have been described as genetic markers of many autoimmune diseases. In turn, MD has been associated with HLA-B*27, HLA-B*44, HLA-B*13, HLA-Cw*07, HLA-DBR1*1602 and HLA-DRB1*1101 in different populations; however these associations have not been replicated in other populations and have no diagnostic usefulness.

Several studies show a high prevalence of autoimmune diseases such as rheumatoid arthritis, systemic lupus erythematosus, ankylosing spondylitis or psoriasis in patients with MD, which suggests a possible altered autoimmune response. For this reason, the systematic study by means of a genotyping microarray (ImmunoChip) of variants located in 186 loci previously associated with 12 autoimmune diseases seems to be one way to investigate the largest possible number of variants per sample, which improves diagnostic performance.

A wide range of both medical and surgical treatments have been proposed for Ménière's disease. All these treatments yield different results with respect to symptom remission and side effects.

Medical treatments include, among others, a low-sodium diet, antihistamines, diuretics, subcutaneous histamine, antivertigo drugs, benzodiazepines and transtympanic therapy, which has gained some popularity in recent years, particularly in the group of patients showing no response to conventional therapies.

Intratympanic injection of ototoxic medications such as gentamicin into the middle ear cavity has been practiced in some centers for some time now. The objective of this procedure would be to produce a medical labyrinthectomy, which would cause the symptoms to improve or disappear. The risk of this procedure is the deterioration of the auditory capacity of the patient, described particularly in those patients subjected to treatment regimens in short time periods. The most widely used agents were streptomycin initially and gentamicin at present.

Surgery, such as labyrinthectomy or vestibular neurectomy, has been used in very serious cases, but it reduces the patient's quality of life because it eliminates not only the symptoms but also the sensory information of the vestibular receptors and brings about chronic disequilibrium. Therefore, treatments currently available for hearing loss can be excessively aggressive and place the patient's hearing at risk. It would therefore be important to have a diagnostic and prognostic marker that allowed evaluating treatment suitability.

However, up until now there has not been any method or biological marker that allowed for an early diagnosis or for establishing a long-term hearing loss prognosis that is useful in classifying patients and makes it easier to make decisions about the treatment and the follow-up of the progression of the disease.

### BRIEF DESCRIPTION OF THE INVENTION

The examples of the invention show that single-nucleotide variants rs9380217, rs4947296 and/or rs886424 in region 6p21.33 are markers that are useful in determining the prognosis of a disease involving sensorineural hearing loss, preferably for the prognosis of the progression of Ménière's disease to Ménière's disease with bilateral involvement.

Therefore, a first aspect of the invention relates to the use of single-nucleotide variants rs9380217, rs4947296 and/or rs886424 to obtain data that is useful in the prognosis of a disease involving sensorineural hearing loss.

In another preferred embodiment of this aspect of the invention, the disease involving sensorineural hearing loss is Ménière's disease.

A second aspect of the invention relates to a method of obtaining data that is useful for the prognosis of a disease involving sensorineural hearing loss in an individual affected by said disease, hereinafter first method of the invention, wherein the method comprises:
a) detecting single-nucleotide polymorphisms or variants rs9380217, rs4947296 and/or rs886424 in a biological sample isolated from the affected individual.

Another aspect of the invention relates to a method for the prognosis of a disease involving sensorineural hearing loss, hereinafter second method of the invention, wherein the method comprises the step of the first method of the invention and further comprises:
b) including the individual (a) in the group of individuals showing progression towards a disease involving bilateral sensorineural hearing loss when said individual presents the homozygous allele with less frequency for rs9380217, rs4947296 and/or rs886424.

In a preferred embodiment of this aspect of the invention, the disease involving sensorineural hearing loss is Ménière's disease or autoimmune inner ear disease. More preferably the disease involving sensorineural hearing loss is Ménière's disease.

In another preferred embodiment of this aspect of the invention, the isolated sample in (a) is peripheral blood, and even more preferably it is genomic DNA obtained from peripheral blood.

In another preferred embodiment of the invention, the individual belongs to a population of European descent, and even more preferably of Spanish descent.

A third aspect of the invention relates to a method, hereinafter third method of the invention, for classifying an individual who suffers or is likely to suffer from a disease involving sensorineural hearing loss, preferably Ménière's disease or autoimmune inner ear disease, into one of two groups, wherein group 1 comprises individuals that can be identified by means of the method according to the first method of the invention as they present the homozygous allele with less frequency for rs9380217, rs4947296 and/or rs886424, and wherein group 2 represents the remaining individuals.

A particular embodiment of this aspect of the invention relates to a composition comprising one or more TWEAK/Fn14 pathway modulator, inhibitor or antagonist compounds, such as an antibody or a fragment thereof capable of binding to the TWEAK ligand (NF-related weak inducer of apoptosis) or to an antibody or fragment thereof capable of binding to Fn-14 (TWEAK receptor), for use in the prophylactic or therapeutic treatment of sensorineural hearing loss, preferably in Ménière's disease, and more preferably in those patients belonging to or classified as group 1 according to the third aspect of the invention. Preferably, said antibody is the BIIB023 antibody (Humanized mAb against tumor necrosis factor (TNF)-like weak inducer of apoptosis (TWEAK) (http://www.biocentury.com/products/biib023). More preferably, this drug is administered systemically or transtympanically.

A fourth aspect of the invention relates to a kit or device, preferably a two-channel microarray, an oligonucleotide DNA chip, a GeneChip or a genotyping DNA chip, suitable for carrying out the method described in any of the preceding aspects, comprising a solid surface, preferably of glass, plastic or silicon, to which there is attached or for which there is designed at least one oligonucleotide complementary to sequence SEQ ID NO: 1 or a fragment thereof comprising the single-nucleotide variant for rs9380217 and optionally oligonucleotides complementary to sequences SEQ ID NO: 2 and/or SEQ ID NO: 3 or fragments thereof comprising the single-nucleotide variant for rs4947296 and/or for rs886424.

Preferably, said kit is a microarray comprising oligonucleotides or single-channel microarrays designed based on the oligonucleotide sequences described in the preceding paragraph. More preferably, said kit is a genotyping DNA chip.

An additional aspect of the present invention relates to the use of a kit or device according to the fourth aspect of the invention for the prognosis of a disease involving sensorineural hearing loss. Preferably, the disease involving sensorineural hearing loss is Ménière's disease or autoimmune inner ear disease. More preferably, the disease involving sensorineural hearing loss is unilateral Ménière's disease.

Another aspect of the invention relates to a computer-readable storage medium comprising program instructions capable of making a computer carry out the steps of any of the methods of the invention.

Another aspect of the invention relates to a transmissible signal comprising program instructions capable of making a computer carry out the steps of any of the methods of the invention.

### DESCRIPTION OF THE DRAWINGS

FIG. 1 shows a scatter plot showing the principal component analysis (PCA) of the genetic markers in the samples of the invention obtained from the Spanish population compared with different HapMap populations. The actual values corresponding to the first three principal components explained most of the sub-structure of the population in this analysis (77.5%). Every person who was not included with the main group (>3 standard deviations away from the center cluster) were excluded from subsequent analysis. This method was able to identify a total of 48 atypical individuals in the case and control cohort of the invention. The X-axis represents principal component 1 (PC1) and the Y-axis represents principal components 3 (PC3) in the samples of the invention obtained from the Spanish population (diamonds), and the main HapMap populations: CEU (squares), CHB+JPB (triangles), MEX (crosses), TSI (asterisks) and YRI (circles).

FIG. 2 shows data about variants rs9380217 and rs4947296 which show that they are in linkage disequilibrium (r2 and base distance between SNPs).

FIG. 3 shows the normalized strength of the signal corresponding to the genotypes of variants rs9380217, rs4947296 and rs886424 by means of the genotyping microarray used in the iSCAN.

FIG. 4 shows receiver operating characteristic (ROC) curves for predicting bilateral SNHL with variants rs9380217, rs886424 and the history of autoimmune disease.

FIG. 5 shows a heat map for the TWEAK/Fn14 pathway which represents the gene expression profile (fold change) of the genes involved for each individual.

FIG. 6 shows a heat map for the protein ubiquitination pathway which represents the gene expression profile (fold change) of the genes involved for each individual.

FIG. 7 shows the gene expression profile (fold change) of the genes involved for each individual.

### DETAILED DESCRIPTION OF THE INVENTION

The present invention provides a method of obtaining data that is useful for the prognosis of Ménière's disease, as well as for assessing the genetic risk of progression of the disease from unilateral to bilateral, which allows optimizing follow-up, treatment planning and genetic counseling. The authors of the present invention have been the first to demonstrate the association of three variants in chromosome 6 located at specific genomic coordinates, which determine a risk of bilateral progression in Ménière's disease. Said variants have a low frequency (0.09 for rs9380217 and rs4947296, 0.05 for rs886424) in the general population and they are very useful in patients for establishing the prognosis of the progression of hearing loss with a high diagnostic yield. Therefore, the presence of allelic variants indicates a high risk for developing bilateral hearing loss in Ménière's disease in the Spanish population and in the population of European descent.

Therefore, a first aspect of the invention relates to the in vitro use of single-nucleotide variants rs9380217, rs4947296 and/or rs886424 as an indicator or as a biomarker; specifically this indicator or biomarker will be used to obtain data that is useful in the prognosis of a disease involving sensorineural hearing loss.

The region where these variants are located corresponds to a binding region of transcription factors CTCF and CEBPB, which act by regulating the expression of genes involved in the immune and inflammatory response.

In another preferred embodiment of this aspect of the invention, the disease involving sensorineural hearing loss is Ménière's disease.

In this specification, “sensorineural hearing loss” is understood as a decrease in hearing level below 30 dB in at least 2 consecutive frequencies at 250, 500, 1000, 2000, 4000 or 8000 Hz in pure-tone audiometry.

Several methods are known in the state of the art for measuring whether or not there is a hearing level loss, and the extent of said loss, such as, for example, but not limited to, pure-tone audiometry or the brainstem auditory evoked potentials.

Here it is determined by means of pure-tone audiometry. The hearing loss can be present in one or both ears and can be sudden or abrupt, rapidly progressive (weeks or months) or slowly progressive (months or years). Sensorineural hearing loss affects the inner ear and occurs when cochlear sensorineural elements or the cochlear nerve are somehow damaged by physical or some other kind of means.

In this specification, “unilateral sensorineural hearing loss” is understood as when the effect occurs in only one ear and “bilateral sensorineural hearing loss” when the effect occurs in both ears.

The cochlea is the fundamental organ of hearing located in the inner ear, in the form of a spiral-shaped duct and containing the organ of Corti, where mechanical vibrations are converted into nerve impulses which reach the brain in order to be identified through the auditory nerve. When organ of Corti and/or auditory nerve cells are affected, sound transmission is cut off. Any of these delicate structures can be affected by different infectious, inflammatory, toxic or degenerative processes, where out of all of them loud noise and degenerative processes are responsible for most cases of perceptive deafness.

According to the affected organ, they can be classified as:
Endocochlear: When the inner ear is affected.
Retrocochlear: When the auditory nerve is affected.

In a preferred embodiment of this aspect of the invention, the sensorineural hearing loss is selected from the list consisting of: sudden hearing loss, rapidly progressive hearing loss, slowly progressive hearing loss, low frequency sensorineural hearing loss, immune-mediated inner ear disease, or any combinations thereof.

In another preferred embodiment, the sensorineural hearing loss is furthermore associated with recurrent attacks of vertigo. In another even more preferred embodiment, the disease associated with sensorineural hearing loss is Ménière's disease. In another more preferred embodiment, the regions of the genome containing the allelic susceptibility variants correspond to the genomic coordinates of Table 1.

Table 1
Genomic coordinates of the variants forming the Ménière's
disease risk diagnostic panel.

| | | Human Genome | | |
|---|---|---|---|---|
| | | Position | Alleles | |
| SNP | Chromosome | (GRCh38/hg38) | (1) | Location |
| rs9380217 | 6 | 31083776 | C/T | Intergenic |
| rs4947296 | 6 | 31090401 | T/C | Intergenic |
| rs886424 | 6 | 30814225 | C/T | Intergenic |

(1) The first allele is the major allele and the reference in the consensus sequence, whereas the second allele is the minor allele and the susceptibility variant. In the context of the present invention, these variants are also defined by a nucleotide or polynucleotide sequence, which are defined in the following sequences:

Chr6:31083776, rs9380217 = SEQ ID NO: 1
TCTTCTCTGCTCTGATTCTTCCTAT[C/T]CCTCAGAAACCCA
AGGCCTCCTTAG
SEQ ID NO 1:
TCTTCTCTGCTCTGATTCTTCCTATTCCTCAGAAACCCAAGGC
CTCCTTAG
chr6:31090401, rs4947296 = SEQ ID NO: 2
CACACAAGAGCAAAAAAGGACGCAA[T/C]GAAATATGGAAAC
TAGTGAATGGAA
SEQ ID NO 2:
CACACAAGAGCAAAAAAGGACGCAACGAAATATGGAAACTAGT
GAATGGAA
Chr6:30814225, rs886424 = SEQ ID NO: 3
CAACTCCAATGTCACCTGCTCCTCA[C/T]CTGTACCCAGTGG
ACTTTTTTGGCA
SEQ ID NO 3:
CAACTCCAATGTCACCTGCTCCTCATCTGTACCCAGTGGACTT
TTTTGGCA

It is noted that the polymorphism rs886424 is located in the strand complementary to the strand from which SEQ ID NO 3 (http://www.ncbi.nlm.nih.gov/snp/?term=rs886424) has been selected.

Variant sequences of any of sequences SEQ ID NO 1 to 3 containing the polymorphism and which are shortened or elongated in some nucleotides would be within the scope of the present invention. In this sense, preferably SEQ ID NO 1 to 3 could be shorted by 5 nucleotides at most, preferably SEQ ID NO 1 and 3 could be shortened by 5 nucleotides at most on the 3′ end and sequence 2 could be shortened by 5 nucleotides at most on the 5′ end.

### METHODS OF THE INVENTION

A second aspect of the invention relates to a method of obtaining data that is useful for the prognosis of a disease involving sensorineural hearing loss in an individual suffering from said disease, hereinafter first method of the invention, wherein said method comprises:
a) detecting the presence or absence of the single-nucleotide polymorphisms or variants rs9380217, rs4947296 and/or rs886424 in a biological sample isolated from said individual.

In other words, step (a) consists of detecting the single-nucleotide polymorphisms or variants which are selected from the list consisting of rs9380217, rs4947296 and/or rs886424. The detection of the presence of the single-nucleotide polymorphisms or variants of step a) can be done by any method known in the state of the art, for example, but not limited to, DNA sequencing, capillary electrophoresis, mass spectrometry, single-strand conformation polymorphism (SSCP), electrochemical analysis, denaturing HPLC in electrophoresis gel, restriction fragment length polymorphisms (RFLPs), and hybridization analysis in genotyping microarrays.

Another aspect of the invention relates to a method, hereinafter second method of the invention, for the prognosis of a disease involving sensorineural hearing loss, wherein said method comprises step (a) of the first method of the invention, and further comprises: b) including the individual (a) in the group of individuals with progression of the disease to bilateral sensorineural hearing loss when said individual presents the homozygous allele with less frequency for rs9380217, rs4947296 and/or rs886424. In other words, they would be genotypes TT for rs9380217, CC for rs4947296 and/or TT for rs886424.

Step (a) of the methods described above can be completely or partially automated, for example, by means of robotic sensing equipment for the detection in step a) or the computerized comparison in step (b).

In a preferred embodiment of this aspect of the invention, the disease involving sensorineural hearing loss is Ménière's disease or autoimmune inner ear disease.

An “isolated biological sample” includes, but is not limited to, cells, tissues and/or biological fluids of an organism obtained by means of any method known by a person skilled in the art. Preferably, the isolated biological sample is peripheral blood and/or comprises peripheral blood mononuclear cells (PBMCs), more preferably mononuclear cells. More preferably, the isolated sample is genomic DNA, and more preferably obtained from peripheral blood.

In another preferred embodiment of the invention, the individual belongs to a population of European descent, and even more preferably of Spanish descent.

In another preferred embodiment of this aspect of the invention, the sensorineural hearing loss is unilateral.

In a preferred embodiment of this aspect of the invention, the sensorineural hearing loss is selected from the list consisting of: sudden hearing loss, rapidly progressive hearing loss, slowly progressive hearing loss, low frequency sensorineural hearing loss, immune-mediated inner ear disease, or any combinations thereof. In another preferred embodiment, the sensorineural hearing loss is further associated with recurrent attacks of vertigo.

As it is used herein, the term “individual” refers to animals, preferably mammals, and more preferably humans. The term “individual” does not intend to be limiting in any aspect, and said individual may be of any age, sex and physical condition.

In the sense used herein, the term “variant” refers to a sequence substantially homologous to the sequence of genes containing the described variants. In general, a variant includes additions, deletions or substitutions of nucleotides. More preferably, the described variants refer to the change or substitution of a nucleotide of the sequence in the human genome of reference in the described positions.

The term “polymorphism” refers to a variation in the nucleotide sequence of a nucleic acid in which every possible sequence is present in a proportion equal to or greater than 1% in a population; in a particular case, when said variation is the nucleotide sequence occurring in only one nucleotide (A, C, T or G), it is referred to as SNP.

The term “gene mutation” refers to a variation in the nucleotide sequence of a nucleic acid in which every possible sequence is present in a proportion less than 1% in a population.

The terms “allelic variant” or “allele” are used interchangeably herein and refer to a polymorphism occurring in one and the locus in one and the same population.

As it is used herein, the term “gene variation” or “gene variant” includes mutations, polymorphisms and allelic variants. A gene variation or variant is found among individuals within populations and among populations within species.

As it is used herein, the term “prognosis” refers to the capacity to discriminate between individuals having a higher or lower risk a suffering from bilateral sensorineural hearing loss, and more preferably, a higher or lower risk of suffering from bilateral Ménière's disease or a higher or lower risk of suffering from autoimmune inner ear disease.

In turn, considering the method of the present invention, other sub-classifications could be established within this main classification, therefore making it easier to choose and establish suitable therapeutic regimens. As understood by a person skilled in the art, this discrimination is not intended to be correct for all the analyzed samples. However, it does require a statistically significant amount of the analyzed samples to be correctly classified. The statistically significant amount can be established by a person skilled in the art by using different statistical tools, for example, but without limitation, by means of determining confidence intervals, determining the p-value of significance, Student's t-test or Fisher's discriminant functions, non-parametric Mann-Whitney measurements, Spearman's correlation, logistic regression, linear regression, area under the ROC curve (AUC). Preferably, confidence intervals are at least 90%, at least 95%, at least 97%, at least 98% or at least 99%. Preferably, the p-value is less than 0.1, 0.05, 0.01, 0.005 or 0.0001. Preferably, the present invention allows correctly detecting predisposition to the disease or the disease in a differential manner in at least 60%, more preferably in at least 70%, much more preferably in at least 80%, or even much more preferably in at least 90% of the subjects of a given analyzed group or population.

Another aspect of the invention relates to a method, third method of the invention, for classifying an individual who suffers or is likely to suffer from a disease involving sensorineural hearing loss, preferably Ménière's disease or autoimmune inner ear disease, into one of two groups, wherein group 1 comprises individuals that can be identified by means of the method according to the first method of the invention as they present the homozygous allele with less frequency for rs9380217, rs4947296 and/or rs886424, and wherein group 2 represents the remaining individuals.

A particular embodiment of this aspect of the invention relates to a composition comprising one or more TWEAK/Fn14 pathway modulator, inhibitor or antagonist compounds, such as an antibody or a fragment thereof capable of binding to the TWEAK ligand (NF-related weak inducer of apoptosis) or to an antibody or fragment thereof capable of binding to Fn-14 (TWEAK receptor), for use in the prophylactic or therapeutic treatment of sensorineural hearing loss in those patients belonging to or classified as belonging to group 1 according to the third aspect of the invention.

### Diagnostic Kit or Device and Uses

Another aspect of the invention relates to a kit or device, hereinafter kit or device for prognosis of the invention, capable of detecting the homozygous allele with less frequency for rs9380217, rs4947296 and/or rs886424. In other words, it is capable of detecting genotypes TT for rs9380217, CC for rs4947296 and/or TT for rs886424. Preferably, said kit comprises at least one oligonucleotide, which includes the sequence and/or the sequence complementary to any of the sequences SEQ ID NO: 1, SEQ ID NO: 2 and SEQ ID NO: 3; the kit preferably comprises at least one complementary oligonucleotide for each of the sequences SEQ. ID NO: 1, SEQ ID NO: 2 and/or SEQ ID NO: 3.

Said kit or device can contain all those reagents needed for analyzing the single-nucleotide polymorphisms or variants of the invention by means of any of the methods existing in the state of the art and/or described herein. The kit can further include, without any type of limitation, buffers, agents for preventing contamination, protein degradation inhibitors, etc. In addition, the kit can include all the supports and containers needed for implementation and optimization thereof. Preferably, the kit further comprises instructions for carrying out any of the methods of the invention.

The oligonucleotide(s) can also be immobilized in spots on a surface (preferably a solid surface). In one of its embodiments, the kit comprises a microarray, or microarray of the invention. An RNA microarray is an array on a solid substrate (usually a glass slide or a silicon thin-film cell) which evaluates large amounts of different RNAs which can be detected by means of specific probes immobilized in spots on a solid substrate. Each spot contains a specific nucleic acid sequence, usually a DNA sequence, as probes (or indicators). Although the number of spots is not limited in any way, there is a preferred embodiment in which the microarray is customized for the methods of the invention. In one embodiment, said customized array comprises 50 spots or less, such as 30 spots or less, including 20 spots or less. Therefore, another aspect of the invention relates to a microarray comprising oligonucleotides designed based on the known sequence of the single-nucleotide polymorphisms or variants of the invention. In another even more preferred embodiment, the known sequence of the single-nucleotide polymorphisms or variants of the invention is nucleotide sequence SEQ ID NO: 1, SEQ ID NO: 2 and SEQ ID NO: 3.

Another aspect of the invention relates to a microarray, hereinafter microarray of the invention, comprising oligonucleotides or single-channel microarrays designed based on nucleotide sequences SEQ ID NO: 1, SEQ ID NO: 2 and/or SEQ ID NO: 3.

Thus, for example, the oligonucleotide sequences are constructed on the surface of a chip by means of the sequential elongation of a growing strand with only one nucleotide using photolithography. The oligonucleotides are therefore anchored at the 3′ end by means of a selective nucleotide activation method, protected by a photolabile reagent, by means of selective light application through a photomask. The photomask can be a physical or virtual photomask.

The oligonucleotide probes can therefore consist of between 10 and 100 nucleotides, more preferably between 20 and 70 nucleotides, and even more preferably between 24 and 30 nucleotides. To quantify gene expression, preferably about 40 oligonucleotides per gene are used.

In situ synthesis on a solid support (for example, glass) could be done by means of ink-jet technology, which requires longer probes. The supports could be, but without limitation, (charged) NC or nylon filters or membranes, silicon, or glass slides for microscopes covered with aminosilanes, polylysine, aldehydes or epoxy. The probe is each of the samples of the chip. The target is the sample to be analyzed: messenger RNA, total RNA, PCR fragment, etc.

The “amplification conditions” or “extension conditions” refer interchangeably to conditions under which a polymerase can add nucleotides to the 3′ end of a polynucleotide. Said amplification or extension conditions are very well known in the art (Sambrook and Russell, 2001. Molecular Cloning: A Laboratory Manual, 3^{rd} Edition, Cold Spring Harbor Laboratory Press; Ausubel et al., Current Protocols in Molecular Biology, 1987-2007, John Wiley & Sons.)

As it is used herein, the term “primer” is known by the person skilled in the art and refers to “oligomeric compounds”, mainly “oligonucleotides” although also “modified oligonucleotides”, which are capable of “priming” DNA synthesis by means of a template-dependent DNA polymerase, i.e., the 3′ end of the oligonucleotide, for example, provides a free 3′—OH group to which additional “nucleotides” can be bound by a template-dependent DNA polymerase, establishing a 3′ to 5′ phosphodiester bond in which deoxynucleoside triphosphates are used and in which pyrophosphate is released. Therefore, except for the intended function, there is no fundamental difference between a “primer”, an “oligonucleotide” or a “probe” according to the invention.

According to the invention, an “oligomeric compound” is a compound consisting of “monomeric units” which can be “nucleotides” alone or “non-natural compounds” (see below), more specifically “modified nucleotides” (or “nucleotide analogs”) or “non-nucleotide compounds”, alone or in combinations thereof. The “oligonucleotides” and “modified oligonucleotides” (or “oligonucleotide analogs”) are subgroups of “oligomeric compounds” in the context of the invention.

In the context of the present invention, the term “oligonucleotide” refers to “polynucleotides” formed from a plurality of “nucleotides” by way of the “monomeric unit”, i.e., an “oligonucleotide” belongs to a specific subgroup of an “oligomeric compound” or “polymeric compound” of ribonucleic acid (RNA) or deoxyribonucleic acid (DNA) with “monomeric units”. Phosphate groups are commonly referred as forming the internucleoside backbone of the “oligonucleotide”. The normal bond of the RNA or DNA backbone is a 3′ to 5′ phosphodiester bond.

The “oligonucleotides” and “modified oligonucleotides” according to the invention can be synthesized as described primarily in the art and in a manner that is known by the person skilled in the art. The methods for preparing oligomeric compounds of specific sequences are known from the art and include, among others, cloning and restriction of suitable sequences and direct chemical synthesis, for example. Such chemical synthesis methods can include, for example, the phosphotriester method described by Narang S. A. et al., 1979, Methods in Enzymology 68.90-98, the phosphodiester method of Brown E. L. et al., 1979, Methods in Enzymology 68, 109-151, the phosphoramidite method disclosed in Beaucage et al., 1981, Tetrahedron Letters 22:1859, and the H-phosphonate method disclosed in Garegg et al., 1985, Chem. Scr. 25:280-282, and the solid support method disclosed in U.S. Pat. No. 4,458,066.

As indicated above, a “nucleic acid”, as well as the “target nucleic acid”, is a polymeric compound consisting of “nucleotides”, as it is known by the person skilled in the art. It is used herein to refer to a “nucleic acid” in a sample to be analyzed, i.e., the presence, absence or amount of which must be analyzed in a sample.

As it is used herein, the expression “complementary (nucleic acid) sequence of a nucleic acid sequence” refers to the fact that the complementary (nucleic acid) sequence referred to is exactly the complement (reverse) of the nucleic acid sequence.

A nucleic acid or polynucleotide sequence can comprise the five biologically occurring bases (adenine, guanine, thymine, cytosine and uracil) and/or bases other than the five biologically occurring bases. These bases can serve different purposes, for example, for stabilizing or destabilizing hybridization; stimulating or inhibiting probe degradation; or as binding points for detectable moieties or shielding moieties. For example, a polynucleotide of the invention can contain one or more modified, non-standard, derivatized base moieties, including, but not limited to, N6-methyl-adenine, N6-tert-butyl-benzyl-adenine, imidazole, substituted imidazoles, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueuosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queuosine, 2-thiocytosine, 5-methyl-2-thiouracil, 2-thiouracil, 2-thiouracil, 4-thiouracil, 5-methyluracil (i.e., thymine), uracil-5-oxyacetic acid methyl ester, 3-(3-amino-3-N-2-carboxypropyl)uracil, (acp3)w, 2,6-diaminopurine and 5-propinyl pyrimidine. Other examples of modified, non-standard or derivatized base moieties can be found in U.S. Pat. Nos. 6,001,611, 5,955,589, 5,844,106, 5,789,562, 5,750,343, 5,728,525 and 5,679,785. Furthermore, a nucleic acid or polynucleotide sequence can comprise one or more modified sugar moieties including, but without limitation, arabinose, 2-fluoroarabinose, xylulose, and hexose.

“Hybridization melting temperature” or “Tm” refers to the temperature under specific conditions at which 50% of a polynucleotide duplex is in single-stranded form and 50% in double-stranded form. There are readily available computer programs for calculating Tm.

Another aspect of the invention relates to the use of a kit or device of the invention, or a microarray of the invention, for the prognosis of a disease involving sensorineural hearing loss. In a preferred embodiment of this aspect of the invention, the disease involving sensorineural hearing loss is Ménière's disease. In another preferred embodiment of this aspect of the invention, the sensorineural hearing loss is unilateral.

In a preferred embodiment of this aspect of the invention, the sensorineural hearing loss is selected from the list consisting of: sudden hearing loss, rapidly progressive hearing loss, slowly progressive hearing loss, low frequency sensorineural hearing loss, immune-mediated inner ear disease, or any combinations thereof. In another preferred embodiment, the sensorineural hearing loss is further associated with recurrent attack of vertigo.

Another aspect of the invention relates to a computer-readable storage medium comprising program instructions capable of making a computer carry out the steps of any of the methods of the invention (of the first or second method of the invention).

In a preferred embodiment of this aspect of the invention, the readable storage medium comprises at least one sequence comprised in the probes of sequence SEQ ID NO: 1, SEQ ID NO: 2 and SEQ ID NO: 3.

In another preferred embodiment of this aspect of the invention, the readable storage medium further comprises the elements required for detecting variants rs9380217, rs4947296 and/or rs886424.

Another preferred embodiment of this aspect of the invention comprises oligonucleotides or single-channel microarrays designed based on at least one known sequence or an mRNA comprised in any of the probes of the invention.

Another aspect of the invention relates to a transmissible signal comprising program instructions capable of making a computer carry out the steps of any of the methods of the invention.

Therefore, for example, the oligonucleotide sequences can be constructed on the surface of a chip by means of the sequential elongation of a growing strand with a single nucleotide using photolithography. Therefore, the oligonucleotides are anchored at the 3′ end by means of a selective nucleotide activation method, protected by a photola bile reagent, by means of selective light application through a photomask. The photomask can be a physical or virtual photomask.

In situ synthesis on a solid support (for example glass) can be done by means of ink-jet technology, which requires longer probes. The supports could be, but without limitation, (charged) NC or nylon filters or membranes, silicon, or glass slides for microscopes covered with aminosilanes, polylysine, aldehydes or epoxy. The probe is each of the samples of the chip. The target is the sample to be analyzed: messenger RNA, total RNA, a PCR fragment, etc.

The terms “polynucleotide”, “oligonucleotide” and “nucleic acid” are used interchangeably herein in reference to polymeric forms of nucleotides of any length, both ribonucleotides (RNAs) and deoxyribonucleotides (DNAs). The terms “amino acid sequence”, “peptide”, “oligopeptide”, “polypeptide” and “protein” are used interchangeably herein and refer to a polymeric form of amino acids of any length, which can be chemically or biochemically modified, coding or non-coding amino acids.

Throughout the description and claims, the word “comprises” and variants thereof do not intend to exclude other technical features, additions, components or steps. For those skilled in the art, other objects, advantages and features of the invention will be inferred in part from the description and in part from the practice of the invention. The following examples and drawings are provided by way of illustration and are not intended to be limiting of the present invention.

### EXAMPLES OF THE INVENTION

The invention will be illustrated below by means of assays conducted by the inventors.

### Materials and Methods

### Subjects and Samples

The present study included an initial cohort of a total of 716 patients with Ménière's disease and 898 controls from Spain. The second replication cohort was formed by 218 patients with bilateral Ménière's disease and 898 controls from Spain. The samples were collected between January 2007 and May 2015.

Inclusion criteria: the patients were diagnosed after the diagnostic scale of the American Academy of Otolaryngology-Head and Neck Surgery (AAO-HNS) (Committee on Hearing and Equilibrium guidelines for the diagnosis and evaluation of therapy in Ménière's disease. American Academy of Otolaryngology—Head and Neck Foundation, Inc. Otolaryngol Head Neck Surg. 1995. 113, 181-185).

Quality controls were used on samples from the individuals to exclude those that did not pass quality filters (elimination of duplicated or familial samples, extreme values in the genetic structure of the population by means of principal component analysis, extreme values in heterozygosity analysis, very low call rate values).

A basic neuro-otological examination including pure-tone audiometry, nystagmus in primary position, gaze-evoked nystagmus, head-shaking nystagmus and standard caloric test was conducted on all patients.

The study was conducted according to the principles of the Declaration of Helsinki for research with human beings. The Clinical Research Ethics Committee of Almeria approved the research protocol. Genomic DNA was isolated from peripheral blood mononuclear cells by means of the QIAamp DNA Mini Kit (Qiagen). All the healthy patients and individuals gave their informed consent for the study.

### Example 1. Genotyping by Means of Immunochip

The ImmunoChip is a genotyping microarray containing 196524 variants (718 insertions/deletions and 195806 SNPs). These variants are concentrated in 186 different loci which have been confirmed in earlier genomic association studies (p<10⁻⁸) in one or more autoimmune diseases. These regions contain common and rare variants (MAF<1%) and include all the known variants in the dbSNP database, the 1000 Genomes project (version from February 2010) and additional sequencing data provided by collaborators.

The samples were genotyped by means of the described IIlumina 200K Infinium high-density array following the manufacturer's protocol in the Centro de Genómica e Investigación Oncológica (Genyo) Pfizer-Universidad de Granada-Junta de Andalucia.

The samples were grouped by means of the Illumina Genome Studio platform algorithm. The genotypes were reviewed and validated manually and single-nucleotide polymorphisms (SNPs) with low quality tracers (call frequency <0.98, cluster separation <0.4) were eliminated. All SNPs with Gencall scores less than 0.15 were excluded. Genotyping quality controls were strict and included the elimination of SNPs with an allele frequency (MAF) of <0.05, not fitted to the Hardy-Weinberg equilibrium (HWE)<0.0001 or with differences in call rate between cases and controls of p<10⁻⁸. After quality controls, 96899 SNPs with MAF>0.05 remained.

The genetic analysis for identifying the associated variants was performed by means of the PLINK computer program v 1.0.7. The principal component analysis (PCA) has allowed defining the groups of populations within the sub-structure of the analyzed samples. For this purpose, the STRUCTURE and EIGENSTRAT programs were used to define the main groups among the data, the proportions of individuals in each cluster and the marginal groups. All the individuals not grouped with the main European ancestry cluster (deviation of >3) were excluded from subsequent analysis.

To evaluate statistical test inflation, after excluding the marginal groups, first the χ² value of the additive genetic model is compared with its theoretical distribution using a Quantile-Quantile (Q-Q) graph to analyze the results observed against the results expected from the statistical Z-test under the null hypothesis of the absence of association throughout the genome. Data analysis is then performed. The additive model was used as a primary inference model, unless the fit testing with respect to this model was significant. In this case, the lower p-value is used for the dominant or recessive additive model.

The approach to correction by multiple comparisons combines statistical methods to reduce the rate of type I error with replication. Logistic regression analysis was performed to determine the association using the PCA of the genotyping array and the ancestry proportion as co-variables, taking into account the structure of the population.

The first phase of these analyses was conducted with genotyping data.

### Results

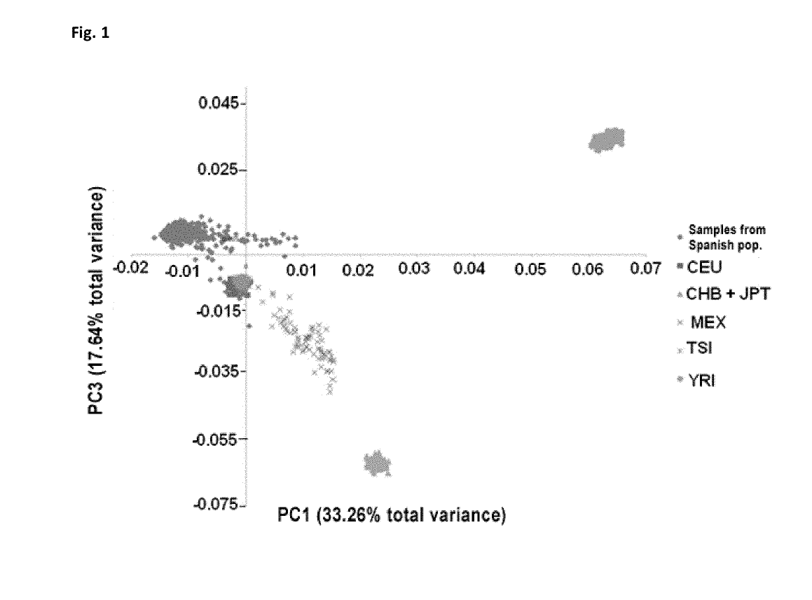

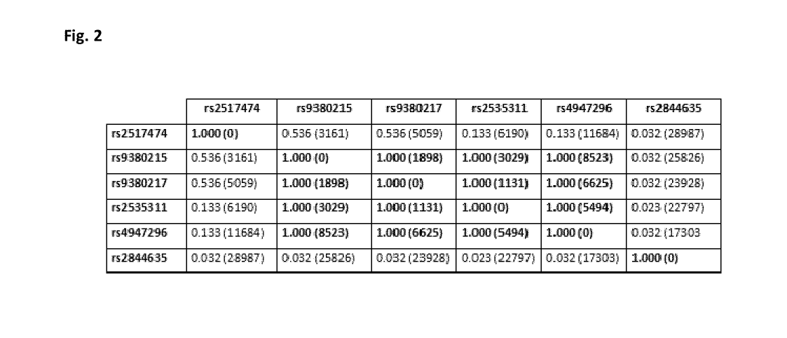

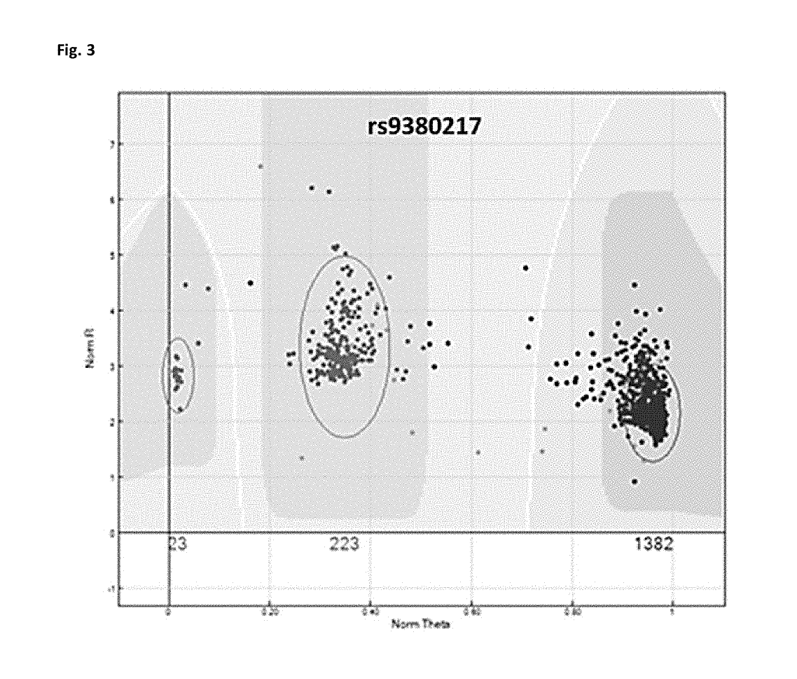

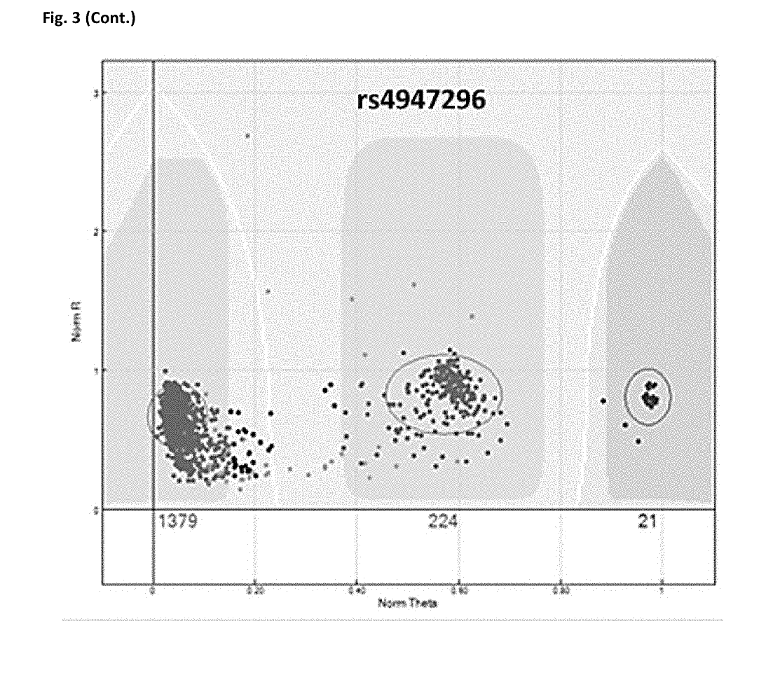

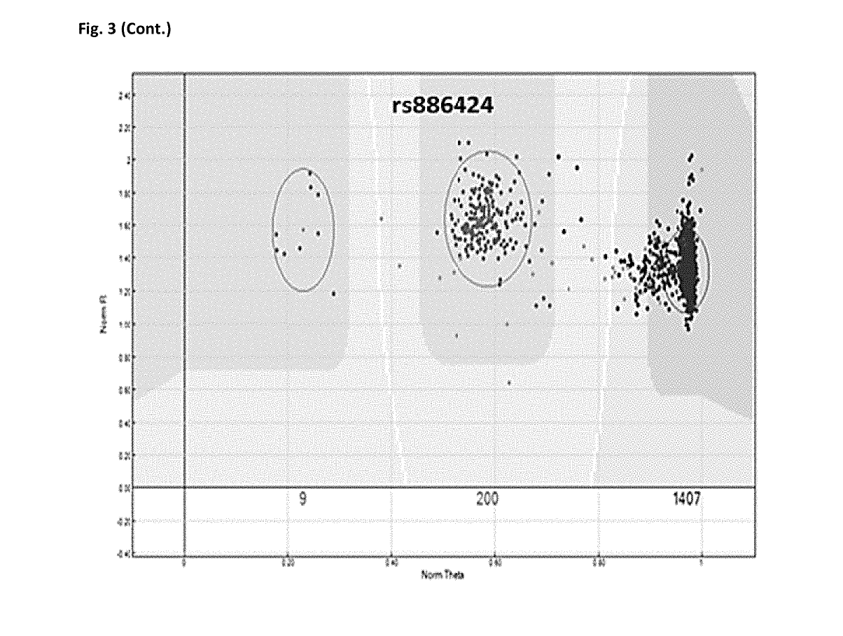

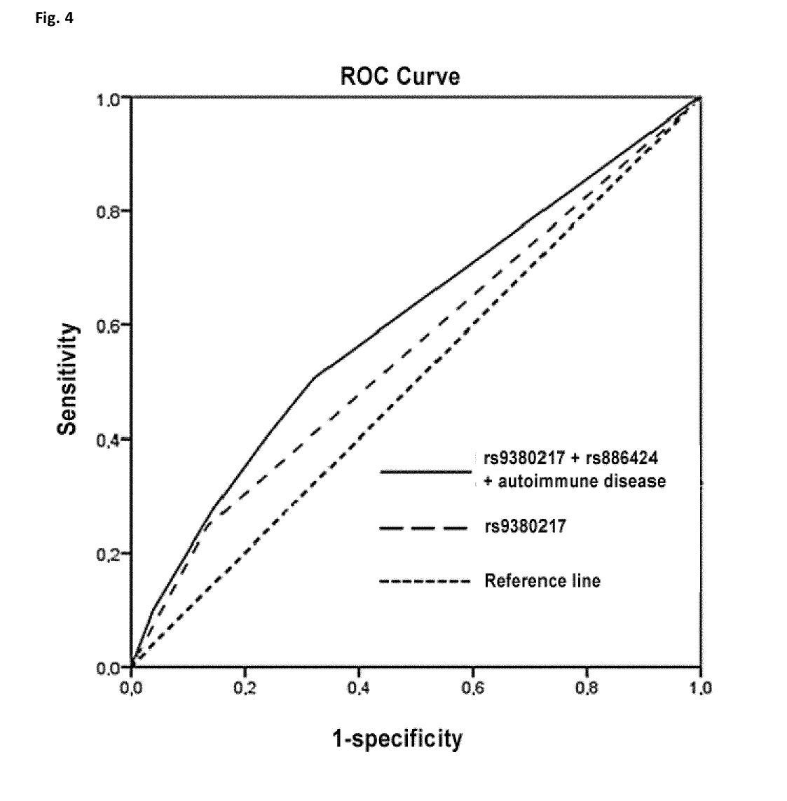

The principal component analysis (PCA) showed that the cases and controls had a similar distribution of the two principal vectors chosen in both groups, which indicates a common genetic background of the individuals in this study and the absence of stratification in the analyzed population (FIG. 1).

No marker reached a significant genomic level (p<10⁻⁸) when comparing all the cases and controls. However, two genomic regions showed a high number of SNPs with p<10⁻⁴ in the bilateral subgroup. These regions were used as candidate loci for replication. The following criteria were used to select the SNPs for replication: p-value, odd ratio, e-QTL (expression quantitative trait loci), MAF (minor allele frequency), information from the Regulome platform (http://regulomedb.org/) and linkage disequilibrium data. SNPs representative of each haploblock were selected to carry out replication: 3 SNPs (rs4988957, rs11465670 and rs4851589) in chromosome 2 and 3 SNPs (rs9380217, rs886424 and rs1150754) in chromosome 6. The results of the replication and meta-analysis are described in Table 2.

A total of 7 SNPs were replicated, only one of which (rs9380217) showing a significant association in the replication cohort. To validate this result another SNP (rs4947296) located in the same haploblock was selected and replicated, confirming the association with region 6p21.33 (chr6: 31081878-31090401) of an approximate size of 10 kb.

The next step was to draw up a logistic regression model for predicting the risk of developing bilateral MD. To decide on the possible factors to be included as co-variables in said model, a random sample including 70% of the individuals for building the model was used, and 30% of the remaining patients were used for validation.

First, the clinical variables significantly associated with bilateral SNHL were identified by means of univariate logistic regression followed by a stepwise regression for selecting the variables that would finally be included in the model. Then, binary logistic regression was used to independently identify the variables associated with bilateral SNHL by means of the Hosmer-Lemeshow goodness of fit. Nagelkerke's R² coefficient of determination summarizes the proportion of variance in the dependent variable associated with the variables. ROC curves were generated to determine the capacity to predict bilateral SNHL in a model made up of rs9380217 and then a model additionally including rs886424 and the clinical variable of autoimmune disease presence. Finally, the areas under the curves were calculated for both models.

The final regression model has the following independent variables: autoimmune disease, allelic variants rs9380217 and rs886424 (Hosmer-Lemeshow test, p=0.980; Table 2). Nagelkerke's R² coefficient of determination indicates that 3.7% of the variance in the variable dependent is associated with the variables. The validity of the predictive model was tested in the remaining 30% of the patients. Precision of the model was 59% in the cases selected for creating the model and 61% in the cases used for validation.

The capacity of the model for predicting progression to bilateral SNHL was evaluated by means of ROC curve tracing using the clinical variables of the model with or without SNP rs9380217. The area under the curve (AUC) was 0.601 (C.I. 95%, 0.562-0.640) for the model including rs9380217, rs886424 and autoimmune disease presence.

The relationship of the genotypes of each variant with the bilateral case/control variable was also evaluated by means of contingency tables, where the result are shown in Table 3.

The results show that allelic variants rs9380217, rs4947296, rs886424 located in region 6p21.33 and the coexistence of autoimmune diseases allow predicting the auditory prognosis of patients with unilateral Ménière's disease progressing to bilateral Ménière's disease.

These findings indicate that patients with unilateral and bilateral hearing loss and Ménière's disease have a different genetic architecture and that explains why autoimmune comorbidity is more common in patients with bilateral sensorineural hearing loss and Ménière's disease.

Autoimmunity has been proposed as a mechanism in patients with bilateral Ménière's disease and although candidate gene studies found that the bilateral disease was associated with allelic variants of genes HLA-DRB1 and PTPN22, these findings have not been replicated in the present study which includes 357 patients with bilateral Ménière's disease and is the largest sample studied up until now.

In general, the data indicates that allelic variants in some genes of the innate immune response, such as TLR10 and NFKB1, could act as regulatory genes capable of modifying the clinical progression of hearing loss in Ménière's disease.

TABLE 2
Replication and meta-analysis results. Cohort-1C (ImmunoChip genotyping): 161 cases/898 controls.
Cohort-TQ (Taqman genotyping): 218 cases/896 controls. Meta-analysis-IC + TQ: 357 cases/1794 controls.

| | | CASES | CONTROLS | P_VALUE | OR (95%) |
|---|---|---|---|---|---|
| rs4988957 | IC | 0.4141 | 0.3576 | 5.24E−02 | 1.27(0.997-1.617) |
| chr2: 102351615 | TQ | 0.3801 | 0.3549 | 1.88E−01 | 1.071(0.930-1.233) |
| | IC + TQ | 0.4065 | 0.3503 | 2.91E−03 | 1.160(1.049-1.831) |
| rs11465670 | IC | 0.1564 | 0.0928 | 5.02E−04 | 1.812(1.291-2.543) |
| chr2: 102417980 | TQ | 0.0867 | 0.0831 | 4.41E−01 | 1.043(0.731-1.489) |
| | IC + TQ | 0.1203 | 0.0872 | 4.52E−03 | 1.380(1.099-1.734) |
| rs4851589 | IC | 0.3374 | 0.2792 | 3.35E−02 | 1.315(1.021-1.693) |
| chr2: 102460685 | TQ | 0.2474 | 0.2578 | 3.61E−01 | 0.960(0.794-1.160) |
| | IC + TQ | 0.2988 | 0.2665 | 4.53E−02 | 1.121(0.988-1.272) |
| rs9380217 | IC | 0.1389 | 0.0734 | 9.30E−05 | 2.034(1.416-2.923) |
| chr6: 31083776 | TQ | 0.1052 | 0.0767 | 3.52E−02 | 1.371(0.998-1.884) |
| | IC + TQ | 0.1291 | 0.0732 | 1.00E−06 | 1.763(1.411-2.202) |
| rs4947296 | IC | 0.1366 | 0.0716 | 8.83E−05 | 2.05(1.423-2.954) |
| chr6: 31090401 | TQ | 0.1071 | 0.0792 | 4.73E−02 | 1.352(0.976-1.874) |
| | IC + TQ | 0.1288 | 0.0741 | 4.00E−06 | 1.741(1.388-2.185) |
| rs886424 | IC | 0.1019 | 0.0509 | 3.55E−04 | 2.111(1.389-3.208) |
| chr6: 30814225 | TQ | 0.0818 | 0.0703 | 2.55E−01 | 1.143(0.807-1.619) |
| | IC + TQ | 0.0949 | 0.0673 | 7.73E−03 | 1.410(1.086-1.832) |
| rs1150754 | IC | 0.1166 | 0.0607 | 2.66E−04 | 2.04(1.381-3.014) |
| chr6: 32082981 | TQ | 0.0892 | 0.0671 | 2.35E−01 | 1.159(0.813-1.653) |
| | IC + TQ | 0.0963 | 0.0706 | 1.34E−02 | 1.364(1.053-1.765) |

TABLE 3
Regression model having the following independent variables:
autoimmune disease, allelic variants of rs9380217 and rs886424
(Hosmer-Lemeshow test, p = 0.980)

| | | | | | 95% C.I. for | | |
|---|---|---|---|---|---|---|---|
| | | | | Exp | EXP (B) | | |
| | B | S.E. | Wald | (B) | Lower | Upper | p value |
| rs9380217 | 0.765 | 0.181 | 17.884 | 2.149 | 1.508 | 3.064 | 2.30E−05 |
| rs886424 | 0.584 | 0.202 | 8.358 | 1.793 | 1.207 | 2.664 | 4.00E−03 |
| History of | 0.522 | 0.197 | 6.996 | 1.685 | 1.145 | 2.48 | 8.00E−03 |
| auto- | | | | | | | |
| immune | | | | | | | |
| disease | | | | | | | |

TABLE 4
Genotype frequencies, odd ratios (OR) with a 95% confidence interval (C.I.) and
sample size (N) of variants rs9380217, rs4947296 and rs886424.

| | | CONTROL (N = 1613) | CASE (N = 335) | | CONTROL (N = 1410) | CASE (N = 266) |
|---|---|---|---|---|---|---|
| rs9380217 | CC | 0.866 (1397) | 0.77 (258) | CC | 0.991 (1397) | 0.97 (258) |
| | CT | 0.134 (216) | 0.23 (77) | TT | 0.009 (13) | 0.03 (8) |
| | | OR (95% C.I.) = 1.930 (1.441-2.585) | | | OR (95% C.I.) = 3.332(1.397-8.120) | |
| | | p-value = 8.00E−06 | | | p-value = 5.00E−3 | |
| rs4947296 | TT | 0.861 (1396) | 0.77 (258) | TT | 0.991 (1396) | 0.97 (258) |
| | CT | 0.134 (217) | 0.23 (77) | CC | 0.009 (13) | 0.03 (8) |
| | | OR (95% C.I.) = 1.920 (1.434-2.571) | | | OR (95% C.I.) = 3.332(1.397-8.120) | |
| | | p-value = 8.00E−06 | | | p-value = 5.00E−3 | |
| | | CONTROL (N = 1618) | CASE (N = 339) | | CONTROL (N = 1423) | CASE (N = 286) |
| rs886424 | CC | 0.875 (1415) | 0.832 (282) | CC | 0.994 (1415) | 0.986 (282) |
| | CT | 0.125 (203) | 0.168 (57) | TT | 0.006 (8) | 0.014 (4) |

| | |
|---|---|
| OR (95% C.I.) = 1.409 (1.023-1.941) | OR (95% C.I.) = 2.509(0.75-8.388) |
| p-value = 3.50E−02 | p-value = 0.122 |

### Example 2. Gene Expression Profile in Mononuclear Cells Conditioned by the Risk/Protector Haplotype

The gene expression profile in mononuclear cells obtained from 5 patients and a control were compared based on the haplotype defined by SNPs rs9380217 and rs4947296 (see table 5), using the IIlumina gene expression array.

TABLE 5
Selected individuals and their corresponding haplotypes for
carrying out the gene expression profile in mononuclear cells
using the IIlumina gene expression array.

| | HAPLOTYPE | |
|---|---|---|
| SAMPLE | rs9380217 | rs4947296 |
| Control | CC | TT |
| Case 1 | CC | TT |
| Case 2 | CC | TT |
| Case 3 | TT | CC |
| Case 4 | TT | CC |
| Case 5 | TT | CC |

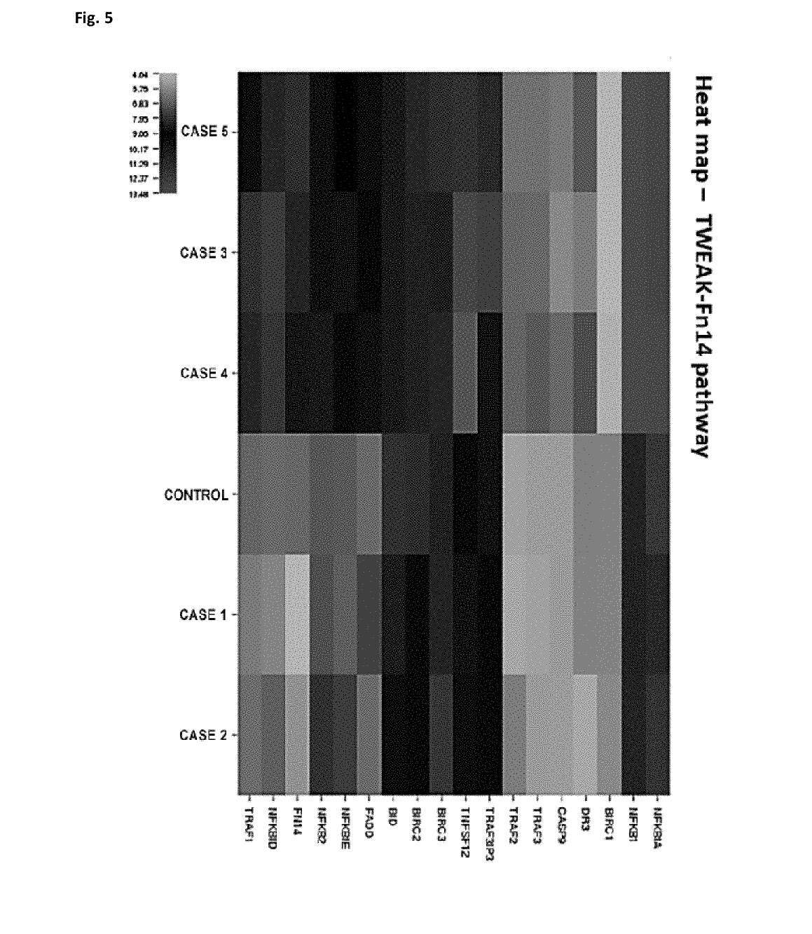

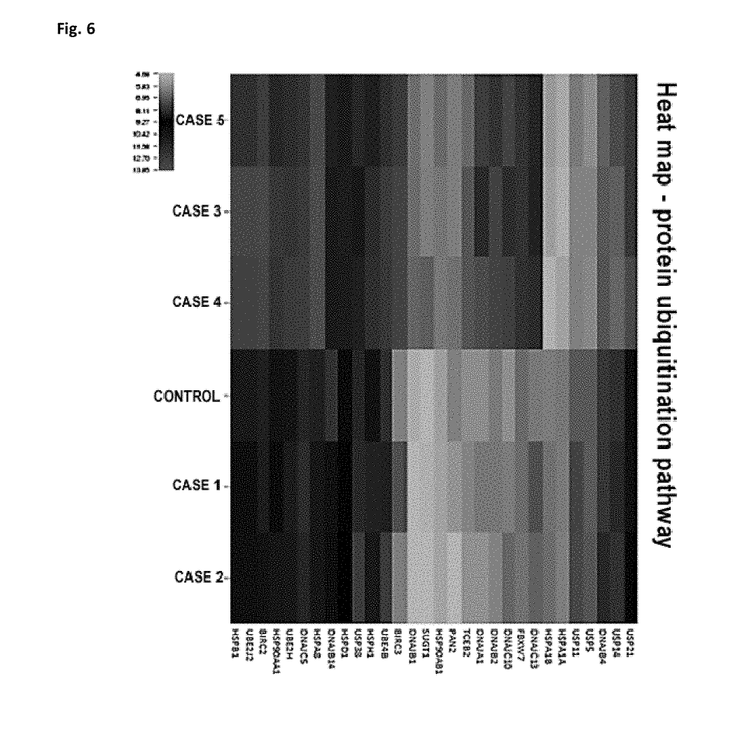

Then, with the genes showing differential expression based on the haplotype, gene interaction network analysis was conducted using IPA (Ingenuity Pathway Analysis) bioinformatic software and several pathways were identified (FIGS. 5 and 6). The NF-related weak inducer of apoptosis (TWEAK)/Fn14 pathway was significantly associated with 11 genes (32% of the genes of the TWEAK/Fn14 pathway) which are over- or under-expressed (p=3.8×10-8). The protein ubiquitination pathway is also associated with 30 genes involved (12% of the genes of the pathway; p=7.9×10-8).

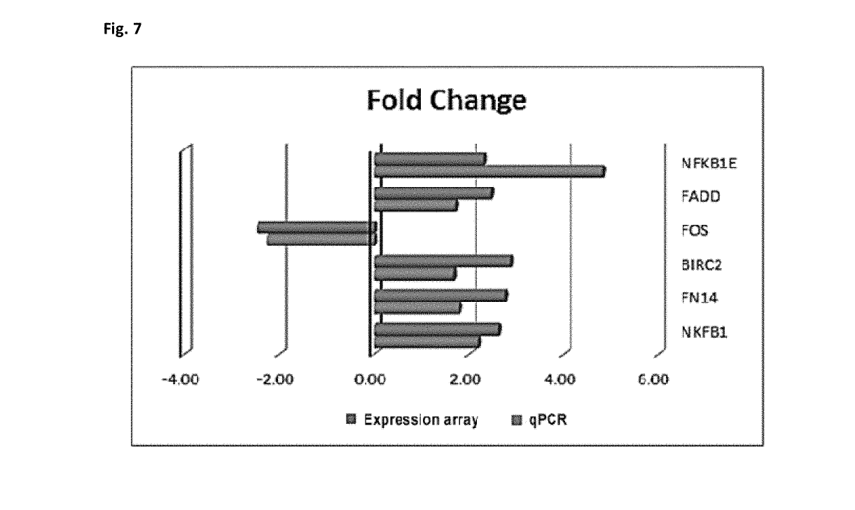

The genes that showed differential expression in the TWEAK/Fn14 pathway (NFKB1, Fn14, BIRC2, FADD, NFKBIE and FOS) were validated by real time PCR (qPCR) (Table 6 and FIG. 7).

TABLE 6
Correlation between gene expression results obtained per
array and validation by qPCR.

| | qPCR | | Expression Array | |
|---|---|---|---|---|
| GENE | Fold Change | p-value | Fold Change | p-value |
| NKFB1 | 2.19 | 3.53E−04 | 2.62 | 2.47E−04 |
| FN14 | 1.78 | 2.06E−03 | 2.75 | 6.41E−03 |
| BIRC2 | 1.66 | 8.56E−03 | 2.86 | 1.05E−03 |
| FOS | −2.27 | 2.57E−02 | −2.46 | 2.17E−03 |
| FADD | 1.70 | 2.57E−02 | 2.45 | 2.35E−03 |
| NFKB1E | 4.81 | 3.98E−04 | 2.30 | 1.97E−03 |

High levels of TWEAK and/or Fn14 have been associated with other autoimmune pathologies, such as rheumatoid arthritis, systemic lupus erythematosus and multiple sclerosis.

Based on these results, a proposal is made to use a pharmaceutical composition comprising an agent that blocks the TWEAK/Fn14 pathway, such as an antibody or fragment thereof capable of binding to the TWEAK ligand (NF-related weak inducer of apoptosis) or to an antibody or fragment thereof capable of binding to Fn-14 (TWEAK receptor), for treating or reducing the seriousness or the effects of patients suffering from sensorineural hearing loss, and particularly of those presenting Ménière's disease with bilateral involvement.

1. In vitro use of the presence of single-nucleotide variant rs9380217 in chromosome 6, the position of which in the human genome (GRCh38/hg38) is 31083776, in a biological sample isolated from an individual, as an indicator for the prognosis of a disease involving sensorineural hearing loss.

2. The in vitro use according to claim 1, which additionally uses single-nucleotide variant rs886424 in chromosome 6, the position of which in the human genome (GRCh38/hg38) is 30814225, in a biological sample isolated from an individual, as an indicator for the prognosis of a disease involving sensorineural hearing loss.

3. The in vitro use according to claim 1 or 2, which additionally uses single-nucleotide variant rs4947296 in chromosome 6, the position of which in the human genome (GRCh38/hg38) is 31090401, in a biological sample isolated from an individual, as an indicator for the prognosis of a disease involving sensorineural hearing loss.

4. A method of obtaining data that is useful for the prognosis of a disease involving sensorineural hearing loss in an individual affected by said disease, wherein said method comprises:
a) detecting the in vitro presence of single-nucleotide variant rs9380217 in chromosome 6, the position of which in the human genome (GRCh38/hg38) is 31083776, in a biological sample isolated from said individual.

5. The method according to claim 4, wherein the in vitro presence of any of the indicators is additionally detected, said indicators being selected from the list comprising:
a) single-nucleotide variant rs886424 in chromosome 6, the position of which in the human genome (GRCh38/hg38) is 30814225, in a biological sample isolated from said individual; and/or
b) single-nucleotide variant rs4947296 in chromosome 6, the position of which in the human genome (GRCh38/hg38) is 31090401, in a biological sample isolated from said individual.

6. A method for the prognosis of a disease involving unilateral sensorineural hearing loss in an individual affected by said disease, wherein said method comprises detecting the presence of the variant defined in claim 4, and further comprises:
b) classifying the individual into the group of individuals showing progression towards a disease involving bilateral sensorineural hearing loss when said individual presents the homozygous allele with less frequency for rs9380217, such that said individual presents the TT genotype for rs9380217.

7. The method for the prognosis of a disease involving unilateral sensorineural hearing loss in an individual affected by said disease according to claim 6, wherein said method further comprises detecting the presence of any of the variants defined in claim 5A and/or 5B, and wherein the individual is classified into the group of individuals showing progression towards a disease involving bilateral sensorineural hearing loss when said individual presents the homozygous allele with less frequency for rs4947296, such that said individual presents the CC genotype for rs4947296 and/or when said individual presents the homozygous allele with less frequency for rs886424, such that said individual presents the TT genotype for rs886424.

8. The method according to any of claims 4 to 7, wherein the disease involving sensorineural hearing loss is Ménière's disease or autoimmune inner ear disease.

9. The method according to any of claims 4 to 8, wherein the isolated sample is genomic DNA obtained from peripheral blood.

10. The method according to any of claims 4 to 9, wherein the individual belongs to a population of European descent, and more preferably of Spanish descent.

11. The method according to any of claims 4 to 5, wherein the sensorineural hearing loss is unilateral.

12. A method for classifying an individual who suffers or is likely to suffer from a disease involving sensorineural hearing loss, preferably Ménière's disease or autoimmune inner ear disease, into one of two groups, wherein group 1 comprises individuals that can be identified by means of the method according to any of claim 6 or 7 as individuals showing progression towards a disease involving bilateral sensorineural hearing loss, and wherein group 2 represents the remaining individuals.

13. A kit or device, preferably a two-channel microarray, an oligonucleotide DNA chip, a GeneChip or a genotyping DNA chip, suitable for carrying out the method described in any of claims 4 to 12, comprising a solid surface, preferably of glass, plastic or silicon, to which there is attached or for which there is designed at least one oligonucleotide complementary to sequence SEQ ID NO: 1 or to a fragment thereof comprising the single-nucleotide variant for rs9380217, and optionally oligonucleotides complementary to sequences SEQ ID NO: 2 and/or SEQ ID NO: 3 or fragments thereof comprising the single-nucleotide variant for rs4947296 and/or for rs88642414.

14. The kit according to claim 13, wherein said kit is a microarray comprising oligonucleotides or single-channel microarrays designed based on the nucleotide sequence described in claim 13.

15. The kit or device according to claim 13, wherein the kit is a genotyping DNA chip.

16. In vitro use of a kit or device according to any of claims 13 to 15, for the prognosis of a disease involving sensorineural hearing loss.

17. Use of a kit or device according to the preceding claim, wherein the disease involving sensorineural hearing loss is Ménière's disease or autoimmune inner ear disease.

18. Use of a kit or device according to any of claim 16 or 17, wherein the disease involving sensorineural hearing loss is Ménière's disease.

19. Use of a kit device according to claims 16 to 18, wherein the sensorineural hearing loss is unilateral.


SEQUENCE LISTING (3 sequences)

SEQ ID NO: 1 - DNA, 51 bp, Homo sapien
tcttctctgctctgattcttcctattcctcagaaacccaaggcctccttag

SEQ ID NO: 2 - DNA, 51 bp, Homo sapien
cacacaagagcaaaaaaggacgcaacgaaatatggaaactagtgaatggaa

SEQ ID NO: 3 - DNA, 51 bp, Homo sapien
caactccaatgtcacctgctcctcatctgtacccagtggacttttttggca